(19) United States
(12) Patent Application Publication
(10) Pub. No.: US 2019/0046649 A1
(43) Pub. Date: Feb. 14, 2019

(54) DELIVERY DEVICE AND USE THEREOF FOR LOADING CELL PLASMA MEMBRANES

(71) Applicants: UNIVERSITY OF MARYLAND, BALTIMORE COUNTY, Baltimore, MD (US); MASSACHUSETTES INSTITUTE OF TECHNOLOGY, Cambridge, MA (US)

(72) Inventors: GREGORY SZETO, BALTIMORE, MD (US); MICHAEL ZHANG, BALTIMORE, MD (US); EMILY SLABY, STREET, MD (US); DARRELL IRVINE, ARLINGTON, MA (US)

(21) Appl. No.: 16/059,156

(22) Filed: Aug. 9, 2018

Related U.S. Application Data
(60) Provisional application No. 62/542,842, filed on Aug. 9, 2017.

(51) Int. Cl. A61K 47/54

(52) U.S. Cl. CPC A61K 47/543; A61P 35/00

(57) ABSTRACT

The present invention provides for an ex vivo delivery platform of rapidly inserting lipid-conjugated molecular ligands into a membrane of cells or biological entities have a lipid membrane by lipid partitioning, termed depoting, wherein the ex vivo delivery platform is a lipid-tailed biomolecule.

### CROSS REFERENCE TO RELATED APPLICATION

This application claims priority to U.S. Provisional Patent Application No. 62/542,842, filed on Aug. 9, 2017, the contents of which are hereby incorporated by reference herein for all purposes.

### BACKGROUND OF THE INVENTION

### Field of the Invention

The present invention relates to a delivery device using lipid-tail molecules for insertion into membranes of contacted cells for delivering biomolecules into and onto such cells.

### Related Art

Cell transfer therapy for cancer therapy and immunotherapy has been recognized using modified stem cells and lymphocytes and has promising potential for treating diverse diseases such as cancer, HIV, and autoimmunity. However, current limitations have made sustaining cures an elusive goal. Such limitations in the clinic include difficulties in harvesting large quantities of antigen presenting cells (APCs) for ex vivo expansion, limited utility of tumor-specific T-cells in treating solid tumors due to adverse immune-related side effects, and suboptimal methods for modification and activation of cells without altering cell state.

Prior work of delivering molecular ligands to cells include lipid-based carriers, such as liposomes and micelles, that couple to immune cells as well as viral vectors. However, with such liposomes and/or micelles the contact with the cell does not necessarily release the enclosed specific cargo into the cell. A major disadvantage of the viral-mediated delivery system relates to the concern about its safety with respect to the possibility of recombination with endogenous virus resulting in a deleterious infectious form of the virus. Further, such a method introduces the potential for genetic mutation/transformation of cells and the inability to inactivate said virus. Others methods include mechanical disturbance of the cell membranes, electroporation, cell penetrating peptides that break or disturb the cell membrane to provide entry into the cell. However, such disturbance can compromise this structure's essential role as a barrier, and this can kill the affected cell. Still further, none of the above methods provide the ability to target a specific section or area of the membrane or cell.

Thus, it would be advantageous to provide a system that overcomes the shortcomings of the prior art methods of delivery.

### SUMMARY OF THE INVENTION

The present invention provides for a delivery platform of rapidly inserting (via spontaneous partitioning by a complex thermodynamic action that increases disorder of lipid acyl chains and/or membrane-thinning) lipid-conjugated molecular ligands into the outer plasma membrane or internal membranes of harvested cells by lipid partitioning, termed depoting. Such delivery platform takes advantage of the biophysical properties of cells or biological entities with lipid containing membranes by directly decorating diverse lipid containing membranes with lipid-conjugated ligands while minimally perturbing cell state or biological entity and eliminating the need for secondary vehicles. Further, homeostatic cell surface turnover or receptor directed internalization can lead to internalization of ligands depoted into cell membrane surfaces, providing an avenue for intracellular trafficking and novel therapy targets toward promoting tumor-specific CD8+ T-cell and CD4+ T-cell immunity as well as non-specific immunomodulation.

In one aspect, the present invention provides for an ex vivo method of introducing a biomolecule cargo into a cell without mechanical disturbance of a cell membrane, the method comprising:

a) contacting the cell with lipid-tailed biomolecule, wherein the lipid-tailed biomolecule comprises:
a lipid component;
a biomolecule cargo for delivery into a cell; and
a linker positioned between and connecting the lipid component and the biomolecule cargo to form the lipid-tailed molecule; and
   b) maintaining the cell in a culture medium a sufficient time for the lipid component of the lipid-tailed molecule to blend with lipids positioned on the cell membrane without mechanical disturbance of the cell membrane and insertion of the biomolecule cargo into the cell.

This ex vivo method provides for expansion or function of the cells with the inclusion of the biomolecule cargo. Generally, such blending of the lipid-tailed molecule to the lipids on the cell membrane can happen as quickly as 15 minutes but noting that such a time can be decreased or extended depending on the amount of molecules to be loaded.

The biomolecule cargo may include but not limited to an immunomodulatory compound, immunoregulatory compound, a molecular adjuvant, nucleosides, nucleotides or oligonucleotides, proteins or peptides. Other types of cargo may be transported with a lipid tail including polysaccharides and other sugars, synthetic inorganic and organic compounds, radioactive compounds or molecules and biological molecules such as growth factors, transcription factors and antibodies.

When deciding on the lipids used in the lipid-tail molecules of the present invention, a review of the lipids of the cell to be modified is helpful. Notably, lipid alterations are associated with a variety of diseases including cancer, obesity, neurodegenerative disorders, cardiovascular pathologies, etc. Thus, when introducing a lipid-tailed molecule of the present invention into a specific cell membrane a review of the lipid structure of such a cell membrane will provide information for the most appropriate lipid for incorporation into the cell membrane. Further, the lipid should be selected to provide a desired and effective period of time on the cell membrane for the transference of the cargo into the cell or for prolonged surface presentation to provide extracellular functions such as signaling, blocking receptors, or enzymatic activity.

In one embodiment, the lipid is tailor made for the specific membrane of the cell. Eukaryotic cells have not only plasma membranes that encase the entire cell but also intracellular membranes that surround various organelles. The plasma membrane includes the endoplasmic reticulum (ER), the nuclear membrane, the Golgi apparatus and lysosomes. Mitochondria and chloroplasts are also surround by membranes.

The lipid-tail molecules disclosed herein typically include a hydrophobic lipid. Examples of preferred lipids include, but are not limited to, fatty acids with aliphatic tails of 8-30 carbons including, linear saturated and unsaturated fatty acids, branched saturated and unsaturated fatty acids, and fatty acids derivatives, such as fatty acid esters, fatty acid amides, and fatty acid thioesters, diacyl lipids, Cholesterol, Cholesterol derivatives, and steroid acids such as bile acids; Lipid A or combinations thereof.

An exemplary lipid is a diacyl lipid or triacyl lipid and preferably having carbon chains between about 12 and 22. Notably, the length of the lipid chains can be varied based upon the lipid content of the cell membrane and needs of the user. It will be understood to those skilled in the art that the lipidated portion of the lipid-tail biomolecule will become incorporated into the phospholipid bilayer that makes up the membrane of the cell to provide a coating to the surface of the cell membrane.

The cell type may include, but is not limited to, a lymphokine-activated killer cell, a dendritic cell, a monocyte, a B cell, a T-cell, a natural killer cell, a neutrophil, an eosinophil, a basophil, a mast cell, a keratinocyte, an endothelial cell, an islet cell, a fibroblast, an osteoblast, a chondrocyte, a muscle cell, a stem cell and a neural cell.

In another aspect the present invention provided for a lipid-tailed biomolecule comprising

(a) a lipid component;
(b) a biomolecule cargo for delivery into a cell; and
(c) a linker positioned between and connecting the lipid component and the biomolecule cargo to form the lipid-tailed biomolecule.

In a still further aspect, the present invention provides for a method of treating a patient for an illness, the method comprising:

a. preparing a lipid-tailed biomolecule for entry into isolated cells or removed cells from the patient:
contacting the isolated cells or removed cells with the lipid-tailed biomolecule, wherein the lipid-tailed biomolecule comprises:
a lipid component;
a biomolecule cargo for delivery into a cell; and
a linker positioned between and connecting the lipid component and the biomolecule cargo;
   b. maintaining the isolated cells or removed cells in an ex vivo culture medium a sufficient time for the lipid component of the lipid-tailed molecule to blend with lipids positioned on the cell membrane without mechanical disturbance of the cell membrane and insertion of the biomolecule cargo into the isolated and removed cells to form activated cells; and
   c. administering or reintroducing an effective amount of the activated cells into the patient.

The illness may include, but is not limited to, cancer, viral infection, autoimmune disease and alloimmune disease.

The removed and isolated cells are generally maintained in a suitable culture medium to provide an environment suitable for their growth and multiplication. Factors affecting growth include nutrients such as carbon, hydrogen, oxygen and nitrogen with smaller quantities of sulphur and phosphorus. The temperature should be maintained between 20 to 45° C. and a pH of from about 6 to 7.5. Most cells need to be supplied with oxygen to grow and all cells require water to grow.

It is therefore an aspect of the invention to provide novel therapeutic cells that have the capability of auto-stimulating themselves and in the process can also stimulate surrounding cells.

Yet another aspect of the present invention is to provide therapeutic cells for the study and treatment of cancer, viral, autoimmune and alloimmune diseases and disorders, as well as any one of a number of conditions in which auto-stimulating cells may be beneficial.

These and other aspects of the invention will be apparent based upon the following description and appended claims.

### BRIEF DESCRIPTION OF THE FIGURES

FIG. 1 shows immune cell depoting of lipid-tailed ligands. A) structure of lipo-ligand; B) schematic of entry; C) FAM labeled; D) depoted in mouse cells; E) MFI of lipo-GP100; F) Lipo-CpG depoted into T-cells; and G) TLR2 ligand depoted into T-cells.

FIG. 2 shows Murine polyclonal T-cells depoted with lipid-tailed TLR2 ligands can activate bystander cells. A) Depoted splenic immune cells: B) Isolated T-cells depoted with TLR2; C) culturing of splenocytes; and D) Microscopy image of Pam2CSK4.

FIG. 3 shows murine polyclonal T-cells depoted with lipo-CpG selectively activate self through autocrine signaling, but exclude activation of bystander cells. A) Bystander CD45.1+ cells; B) Wild type deported B-cells.

FIG. 4 shows murine B-cells are comparable to bone marrow derived cells (BMDCs) as antigen presenting cells (APCs) when presenting lipo-GP100 antigen in a MHCI-restricted manner. A) Resting and active B-cells were depoted; B) LipoGP100 depoted resting B-cells; C) Difference between depoted and pulsed.

FIG. 5 shows murine B-cells as antigen presenting cells (APCs) present to cognate CD8⁺ T-cells in a delayed and prolonged manner when antigen is delivered by lipo-PEG-GP100 (lipo-GP100) depoting. Shows the difference between A) endocytosis, B) direct binding or C) depoting.

FIG. 6 shows Lipo-TLR ligand depoted into murine T-cells enhance cell proliferation. A) Histograms showing effectiveness of depoting; B) Quantification of division index.

FIG. 7 shows Lipo-TLR9 ligand and TLR2 ligand combination-depoted into murine immune cells to overcome suppressive tumor cell signaling. A) Flow cytometry histogram; B) Quantification of CD8+ T-cells; C) Production of IL-2.

FIG. 8 shows structure of lipo-PEG-GP100 and depoting results of such lipid-tailed molecule. A) Diacyl (C18) lipid conjugated ligand; B) FAM labeled concentration of lipo-GP100; C) Median fluorescence intensity of Lipo-GP100.

FIG. 9 shows the TLR2 ligand depoted into CD3+ T-cells and delivered paracrine signaling. A) Depoting into CD3+CD8+ or CD3+CD8-T-cells; B) TLR2 ligand depoted into CD8+ T-Cells; C) MFI intensity.

FIG. 10 shows lipo-Av depots in B and T cells deliver autocrine and paracrine stimulation. A) B-cells depoted with lipo-CpG; B) CD8+ T-cells and B-cells depoted with Pam2; C) Histogram showing CFSE dilution.

FIG. 11 shows that TLR2 ligand depoted T cells activate neighboring immune cells.

FIG. 12 shows a schematic of HIV-specific CTLs with enhanced “shock and kill” capacity through depoting of engineered lipid-tailed proteins and peptides.

### DETAILED DESCRIPTION OF THE INVENTION

### Definitions

An immunostimulatory oligonucleotide, as used herein, is an oligonucleotide that can stimulate (e.g., induce or enhance) an immune response.

As used herein, CG oligodeoxynucleotides (CG ODNs) are short single-stranded synthetic DNA molecules that contain a cytosine nucleotide (C) followed by a guanine nucleotide (G).

By “immune cell” is meant a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes).

The term “T cell” refers to a CD4+ T cell or a CD8+ T cell. The term T cell includes TH1 cells, TH2 cells and TH17 cells.

The term “T cell cytotoxicity” includes any immune response that is mediated by CD8+ T cell activation. Exemplary immune responses include cytokine production, CD8+ T cell proliferation, granzyme or clearance of an infectious agent.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

The terms “individual, “subject,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject, rodents, such as mice and rats, and other laboratory animals.

As used herein, the term “polypeptide” refers to a chain of amino acids of any length, regardless of modification (e.g., phosphorylation or glycosylation).

The term “effective amount” or “therapeutically effective amount” means a dosage sufficient to provide treatment for a disorder, disease, or condition being treated, to induce or enhance an immune response, or to otherwise provide a desired pharmacologic and/or physiologic effect. The precise dosage will vary according to a variety of factors such as subject-dependent variables (e.g., age, immune system health, etc.), the disease, the disease stage, and the treatment being effected.

The present invention relates to the successful intracellular delivery of agents (cargo) not naturally taken up by cells that is achieved by exploiting the natural process of intracellular membrane fusion. The membrane barrier can be overcome by associating these agent substances in conjugates with the lipids that closely resemble the lipid composition of natural cell membranes. These lipids are able to fuse with the cell membranes on contact, and in the process, the associated cargo substances are delivered intracellularly. Lipid conjugates can not only facilitate intracellular transfers by fusing with cell membranes but also by overcoming charge repulsions between the cell membrane and the cargo to be inserted.

The lipid-tailed molecules of the present invention comprise a lipid component in combination with a linker/cargo for delivery and insertion into the cell.

Lipids make up the bulk of biological membranes, however, different cells contain hundreds of different lipid species that can be categorized into three main classes: glycerophospholipids, sphingolipids, and sterols. Glycerophospholipids have a polar head group and two hydrophobic hydrocarbon tails. Gycerophospholipds are molecules composed of glycerol, a phosphate group and two fatty acid chains. The tails are usually fatty acids, and they can differ in length (they normally contain between 14 and 24 carbon atoms). One tail usually has one or more cis-double bonds (i.e., it is unsaturated), while the other tail does not (i.e., it is saturated). Furthermore, additional complexity of eukaryotic lipids is generated by the many possible modifications of the hydrophilic head groups and the hydrophobic hydrocarbon tails. Their hydrophobic portion is a diacylglycerol (DAG), which contains saturated or cis-unsaturated fatty acyl chains of varying lengths. For example the head group of a glycerophospholipid can be modified by the addition of various chemical moieties onto the sn-3 position of the glycerol backbone, leading to a number of different phosphatidyl lipids, such as phosphatidylcholine (PC), -ethanolamine (PE), -serine (PS), -glycerol (PG), -inositol (PI), or the unmodified phosphatidic acid (PA). The fatty acid chains positions can be variable in terms of length and numbers of double bonds (degree of saturation), and the linkage to the glycerol backbone can also be varied by ester, alkyl ether, or alkenyl ether bonds.

The sphingolipids constitute another class of structural lipids. Their hydrophobic backbone is ceramide (Cer). The major sphingolipids in mammalian cells are sphingomyelin (SM) and the glycosphingolipids (GSLs), which contain mono-, di- or oligosaccharides based on glucosylceramide (GlcCer) and sometimes galactosylceramide (GalCer). Gangliosides are GSLs with terminal sialic acids. Sphingolipids have saturated (or trans-unsaturated) tails so are able to form taller and narrower cylinders than PtdCho lipids of the same chain length and pack more tightly, adopting the solid ‘gel’ or so phase; they are also fluidized by sterols.

Sterols are the major non-polar lipids of cell membranes: cholesterol predominates in mammals.

In some embodiments, the lipid is a diacyl lipid or two-tailed lipid. In some embodiments, the tails in the diacyl lipid contain from about 8 to about 30 carbons and can be saturated, unsaturated, or combinations thereof.

Generally, the cargo can include therapeutic, prophylactic or diagnostic agents. The therapeutic and diagnostic agents can be nucleosides, nucleotides or oligonucleotides, proteins or peptides, polysaccharides and other sugars, synthetic inorganic and organic compounds, metals or radioactive compounds or molecules. The cargo of the lipid-tailed molecule disclosed may include a molecular adjuvant such as an immunostimulatory oligonucleotide, or a peptide antigen. However, the cargo can also be other oligonucleotides, peptides, Toll-like receptor agonists or other immunomodulatory compounds, dyes, MRI contrast agents, fluorophores or small molecule drugs that require efficient trafficking into a cell.

In some embodiments, the immunostimulatory oligonucleotide can serve as a ligand for pattern recognition receptors (PRRs). Examples of PRRs include the Toll-like family of signaling molecules that play a role in the initiation of innate immune responses and also influence the later and more antigen specific adaptive immune responses. Therefore, the oligonucleotide can serve as a ligand for a Toll-like family signaling molecule, such as Toll-Like Receptor 9 (TLR9).

In some embodiments, an immunostimulatory oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CG dinucleotide(s), as well as the precise base sequences flanking the CG dimers.

Other PRR Toll-like receptors include TLR3, and TLR7 which may recognize double-stranded RNA, single-stranded and short double-stranded RNAs, respectively, and retinoic acid-inducible gene I (RIG-1)-like receptors, namely RIG-I and melanoma differentiation-associated gene 5 (MDAS), which are best known as RNA-sensing receptors in the cytosol. Therefore, in some embodiments, the oligonucleotide contains a functional ligand for TLR3, TLR7, or RIG-I-like receptors, or combinations thereof.

In some embodiments, the cargo is single-stranded DNA, single-stranded RNA, or double-stranded RNA. The oligonucleotide can be between 2-100 nucleotide bases in length, wherein the 3′ end or the 5′ end of the oligonucleotides can be conjugated to the linker. The oligonucleotides can be DNA or RNA nucleotides which typically include a heterocyclic base (nucleic acid base), a sugar moiety attached to the heterocyclic base, and a phosphate moiety which esterifies a hydroxyl function of the sugar moiety. The principal naturally-occurring nucleotides comprise uracil, thymine, cytosine, adenine and guanine as the heterocyclic bases, and ribose or deoxyribose sugar linked by phosphodiester bonds.

In some embodiments, the oligonucleotides are composed of nucleotide analogs that have been chemically modified to improve stability, half-life, or specificity or affinity for a target receptor, relative to a DNA or RNA counterpart. The chemical modifications include chemical modification of nucleobases, sugar moieties, nucleotide linkages, or combinations thereof. As used herein “modified nucleotide” or “chemically modified nucleotide” defines a nucleotide that has a chemical modification of one or more of the heterocyclic base, sugar moiety or phosphate moiety constituents. In some embodiments, the charge of the modified nucleotide is reduced compared to DNA or RNA oligonucleotides of the same nucleobase sequence. For example, the oligonucleotide can have low negative charge, no charge, or positive charge.

Peptide cargos can include an antigenic protein or polypeptide. The peptide can be 2-100 amino acids (aa), including for example, 5 amino acids, 10 amino acids, 15 amino acids, 20 amino acids, 25 amino acids, 30 amino acids, 35 amino acids, 40 amino acids, 45 amino acids, or 50 amino acids. In some embodiments, a peptide can be greater than 50 amino acids. In some embodiments, the peptide can be >100 amino acids. A protein/peptide can be linear, branched or cyclic. The peptide can include D amino acids, L amino acids, or a combination thereof. The peptide or protein can be conjugated to the polar block or lipid at the N-terminus or the C-terminus of the peptide or protein.

The protein or polypeptide can be any protein or peptide that can induce or increase the ability of the immune system to develop antibodies and T-cell responses to the protein or peptide. Examples of specific peptide and protein antigens that can be used in the lipid-tailed molecules disclosed herein are discussed in more detail below with respect to preferred antigens that can be used ex vivo for insertion into cells.

Tumor antigen cargo are useful as targets for antibody-conjugated chemotherapeutic or cytotoxic agents. These are not specific markers for tumor cells in most cases; rather, they are overexpressed on tumor cells compared with normal tissue.

In some embodiments, the lipid-tailed molecules disclosed herein include a detection label, for example, a fluorophore such as fluorescein or rhodamine, Alexa Fluor dyes, DyLight Fluor dyes, Quasar and Cal Fluor dyes, cyanine dyes (Cy3, Cy5, Cy5.5, Cy7) or other fluorescent dyes. The label can be the cargo, or can be in addition to a cargo.

The linker is a molecule that effects access of the lipid of the lipid-tailed molecule to the membrane of cells. The length and composition of the linker can be adjusted based on the lipid and cargo selected. Suitable linkers include, but are not limited to, one or more ethylene glycol (EG) units, more preferably 2 or more EG units (i.e., polyethylene glycol (PEG)), cell-triggered linkers, tissue or organ response linkers, degradable linkers, reactable linkers, etc. Notably, by selectively choosing a specific linker, the cargo is the released into the cell.

For example, in some embodiments, a peptide conjugate includes a protein or peptide (e.g., peptide antigen) and a hydrophobic lipid linked by a polyethylene glycol (PEG) molecule or a derivative or analog thereof may be used. The precise number of EG units depends on the lipid and the cargo, however, typically, a polar block can have between about 1 and about 100, between about 20 and about 80, between about 30 and about 70, or between about 40 and about 60 EG units. In some embodiments, the polar block has between about 45 and 55 EG, units. For example, in one preferred embodiment, the polar block has 45 EG units.

The lipid tail is coupled to the linker and such bonding may include ester bond linkages, amide bond linkages, thioester bond linkages, or combinations thereof. The lipid and the linker/cargo are covalently linked. Such bonding may be a covalent bond that can be either a non-cleavable linkage or a cleavable linkage. The non-cleavable linkage can include an amide bond or phosphate bond, and the cleavable linkage can include a disulfide bond, acid-cleavable linkage, ester bond, anhydride bond, biodegradable bond, or enzyme-cleavable linkage.

In a preferred embodiment, the lipid-tailed molecules are administered in vitro or ex vivo to the cells. For example, cells are removed from the body, treated with the lipid-tailed molecules, alone or in combination with an adjuvant, and then reintroduced into the patient to be treated. In ex vivo methods, the desired cells are isolated from a sample of a patient's blood or tissues and combined with the lipid-tailed molecule. Additional manipulations of the present cells are possible during the ex vivo step, for example, cytokine treatment and amplification of cell numbers.

The present invention is further directed to methods for treating an illness wherein the method generally comprise administering to a patient an effective amount of the cells prepared ex vivo as described above.

The present invention using a lipid-tailed biomolecule presents a promising approach for 1) utilizing a non-classical but physiologically abundant APC such as B-cells for cross-presentation of antigen, 2) promoting activation of peripheral immune cells and functionalized T-cells through paracrine and autocrine signaling of lipo-TLR agonists, and 3) increasing proliferation of functionalized T-cells under immune-suppressive tumor environments.

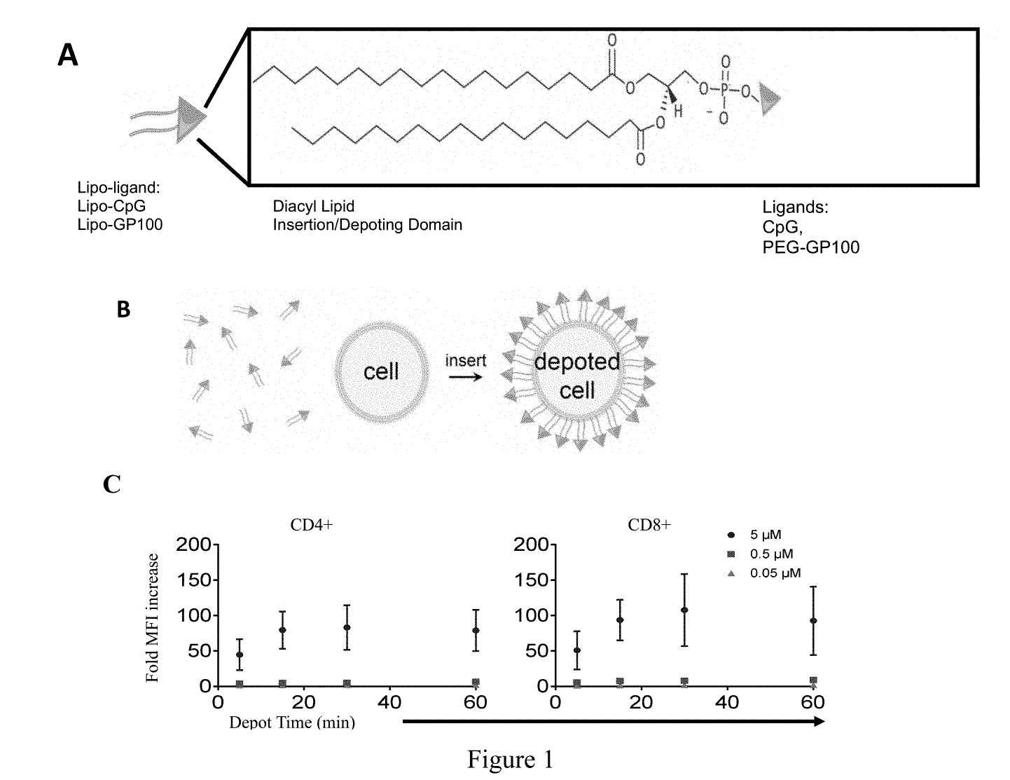

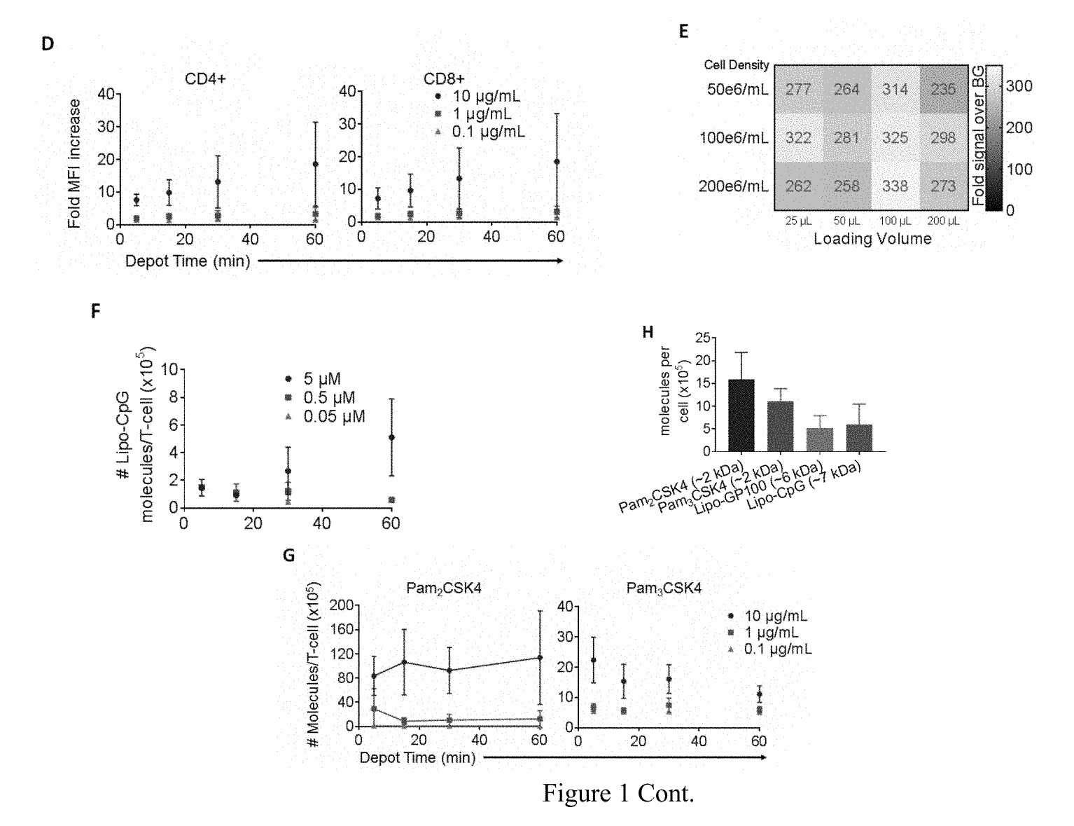

FIG. 1 shows immune cell depoting of lipid-tailed ligands. A) Structure of synthesized lipo-ligand, which are diacyl 18-carbon tails conjugated to: unmethylated CpG oligonucleotide (TCCATGACGTTCCTGACGTT) (SEQ ID NO: 1), or a poly(ethylene) glycol (PEG) linker plus a melanoma antigen, GP100₂₅₋₃₃ (KVPRNQDWL) (SEQ ID NO: 2). B) Schematic of lipo-ligand depoting into cell membrane surface. C-D, CD4+ and CD8+ T-cells are identified by flow cytometry and median fluorescence intensity (MFI) of labeled ligand is determined on each cell subset as a function of time and ligand concentration. C) Fluoroscein (FAM)-labeled lipo-CpG, a TLR9 ligand, depoted into murine splenic immune cells. D) Pam₂CSK4, a TLR2 ligand, depoted into murine splenic immune cells. E) MFI of lipo-GP100 is determined by flow cytometry as a function of cell density and depot volume. F-G, Isolated T-cells are depoted by varying depot time or ligand concentration, and quantified as average number of ligand per T-cell by enzyme-linked immunosorbant assay (ELISA) F) Lipo-CpG depoted into isolated T-cells. G) Pam₂CSK4 and Pam₃CSK4, another TLR2 ligand, respectively depoted into isolated T-cells. H) Average number of each respective ligand per splenic immune cell after 1 hour depot. n=3-5 independent experiments. Data depict m±s.d.

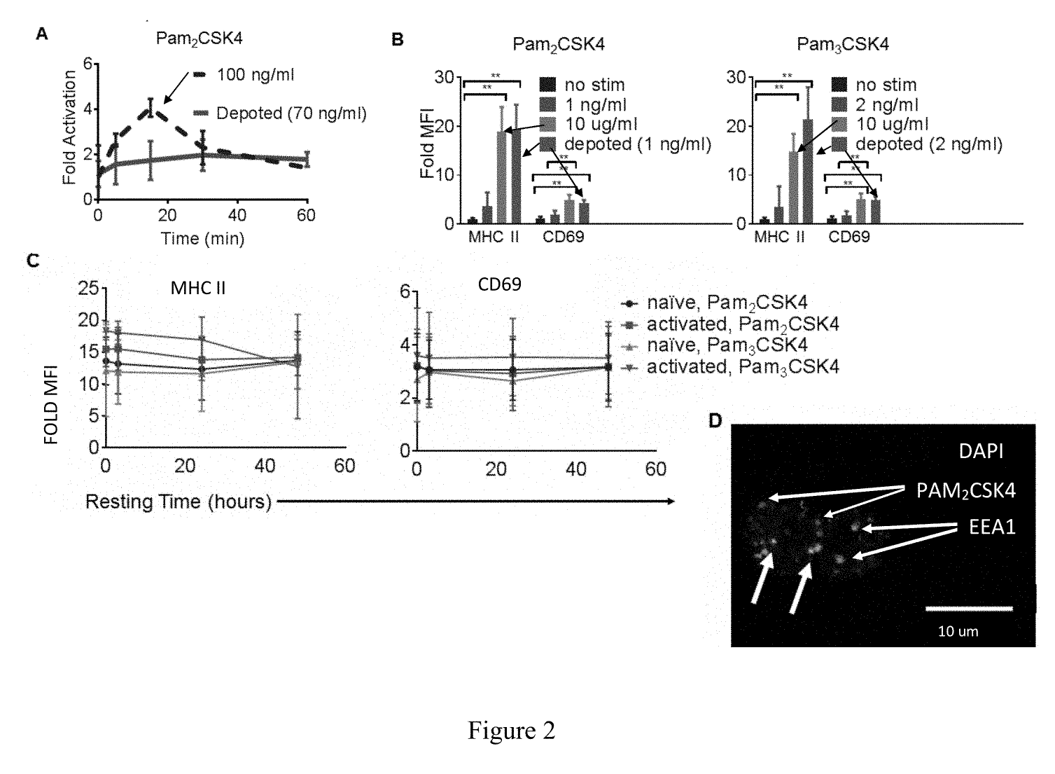

FIG. 2 shows murine polyclonal T-cells depoted with lipid-tailed TLR2 ligands can activate bystander cells. A) Splenic immune cells were depoted with 10 μg/ml of Pam₂CSK4 for 1 h. Depoted cells were resting for up to 1 h versus nondepoted cells with addition of 100 ng/ml of soluble Pam₂CSK4. Phosphorylated p38 was measured compared to negative controls by western blotting. n=2 independent experiments. Data depict m±s.d. B) Isolated T-cells were depoted with TLR2 ligand and MFI of activation markers, MHCII and CD69, on bystander B-cells were measured after 2-day culture by flow cytometry and normalized to unstimulated B-cells (fold MFI). n=5-6 independent experiments, one-way ANOVA with Kruskal-Wallis test. Data depict m±s.d. C) Splenocytes were cultured in 2 μg/ml of Concanavalin A and 10 ng/ml of IL-7 for 2 days, and isolated T-cells were then TLR2 ligand-depoted for 1 hour. Depoted T-cells were then resting for 0 h, 3 h, 24 h, or 48 h, before T-cells were chemically fixed and co-cultured for 2 days with CD45.1⁺ bystander B-cells. Bystander B-cells were used as a reporter to determine TLR2 ligand surface persistence on depoted T-cells by their fold MFI of MHCII and CD69. n=5 independent experiments. Data depict m±s.d. D) Microscopy image of Pam₂CSK4 colocalizing with early endosome antigen 1 (EEA1) 24 h post-depoting, as indicated by arrows.

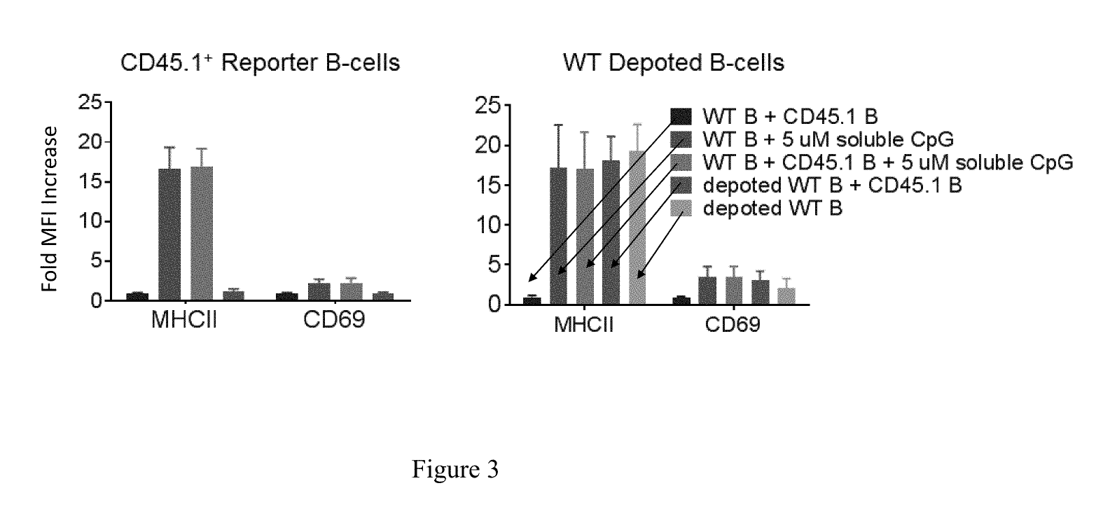

FIG. 3 shows murine polyclonal T-cells depoted with lipo-CpG selectively activate self through autocrine signaling, but exclude activation of bystander cells. Isolated wildtype (WT) B-cells were depoted with lipo-CpG TLR9 ligand and co-cultured with CD45.1⁺ bystander B-cells. Activation markers, MHCII and CD69, on both depoted and bystander B-cells were measured after 2-day culture by flow cytometry and normalized to unstimulated B-cells (fold MFI). n=4 independent samples, one-way ANOVA with Geisser-Greenhouse correction. **p=0.01 between indicated conditions. Data depict m±s.d.

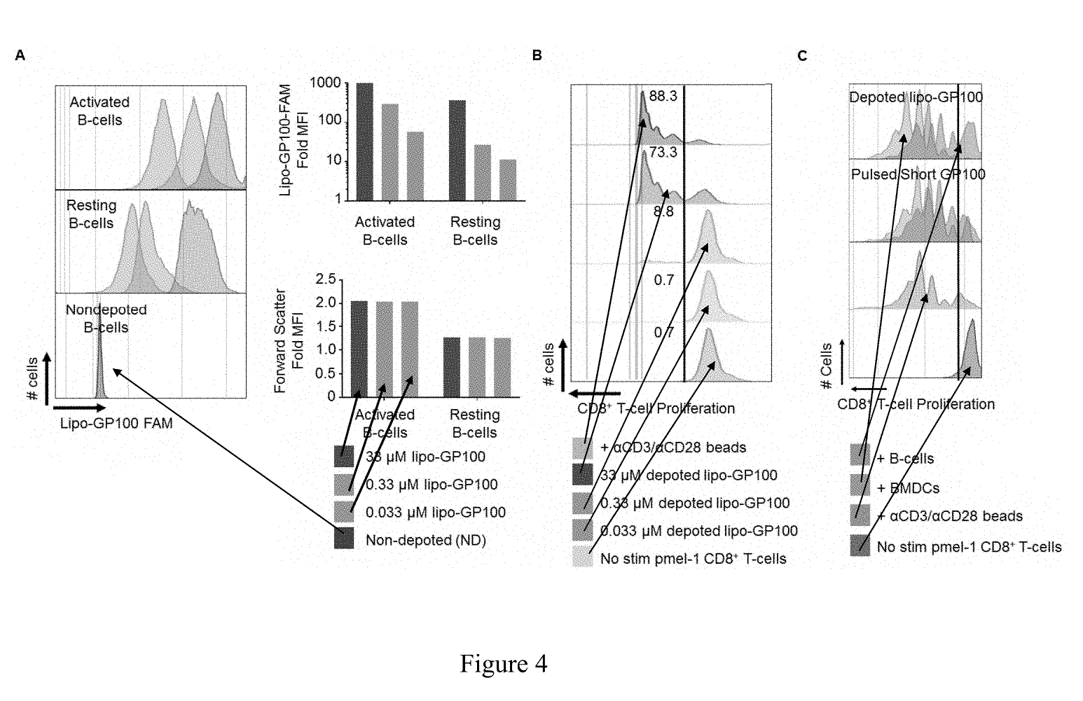

FIG. 4 shows murine B-cells are comparable to bone marrow derived cells (BMDCs) as antigen presenting cells (APCs) when presenting lipo-GP100 antigen in a MHCI-restricted manner. A) Activated B-cells were isolated from splenocytes and cultured with 1 μg/ml TLR7 ligand R848 and αIgM antibody for 2 days. Resting versus activated B-cells were depoted with lipo-GP100-FAM for 1 hour. Lipo-GP100-FAM and forward scatter fold increase was measured by flow cytometry. B) LipoGP100-depoted resting B-cells presented to and proliferated cognate CD8⁺ T-cells at 33 μM of 1 h depoted lipo-GP100 after 3-day culture as measured by CFSE dilution with flow cytometry. Indicated values are percent CD8⁺ T-cells that divided at least once. C) BMDCs were cultured with 20 ng/ml of GM-CSF for 7 days before harvested and either depoted with 33 μM of lipo-GP100 or pulsed with 33 μM of short GP100 (KVPRNQDWL) (SEQ ID NO: 2) peptide, along with resting B-cells. These modified APCs were co-cultured with pmel-1 GP100-specific CD8⁺ T-cells for 3 days to determine pmel-1 T-cell proliferation by flow cytometry. n=1 independent experiment.

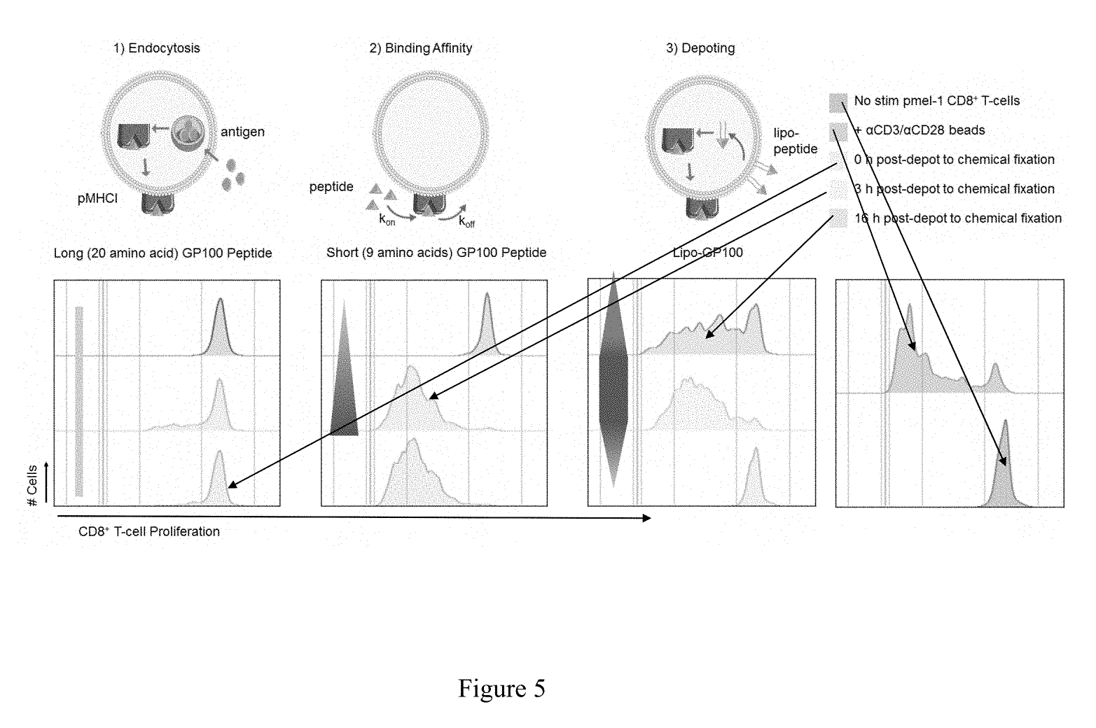

FIG. 5 shows murine B-cells as antigen presenting cells (APCs) present to cognate CD8⁺ T-cells in a delayed and prolonged manner when antigen is delivered by lipo-PEG-GP100 (lipo-GP100) depoting. Antigen processing in APC B-cells by A) endocytosis, B) direct binding affinity on APC surface, or C) depoting. 33 uM of indicated GP100 antigen is conferred to APC B-cells for 1 h, then processed by B-cells in aforementioned antigen processing mechanisms for 0 h, 3 h, or 16 h, before B-cells are chemically fixed and cultured with cognate CD8⁺ T-cells for 3 days to determine T-cell proliferation. n=1 independent experiment.

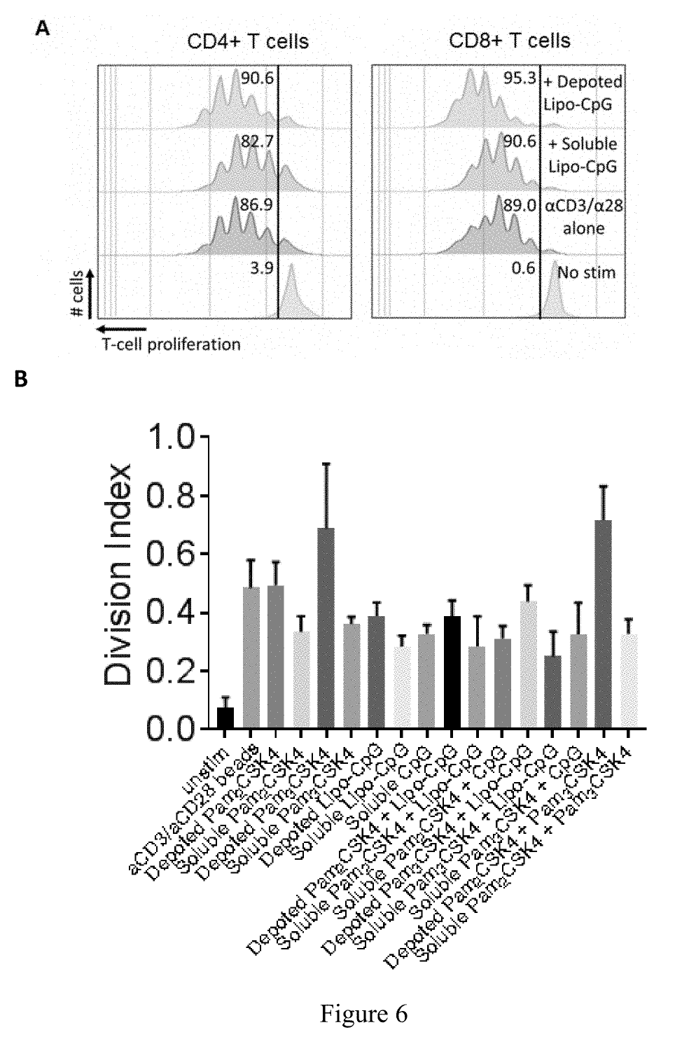

FIG. 6 shows lipo-TLR ligand depoted into murine T-cells enhance cell proliferation. A) Representative histograms showing enhanced T-cell proliferation of depoted CD8⁺ T-cells after 3-day stimulation with ratios of 1:1 αCD3/CD28 beads. T-Cells were cultured with 5 μM of soluble lipo-CpG or depoted with lipo-CpG, and CFSE dilution was measured by flow cytometry. B) Quantification of division index of CD4⁺ T-cells as measured by CFSE dilution from flow cytometry. n=3 independent experiments, one-way ANOVA with Krustal-Wallis test. *p<0.05 between the unstim condition.

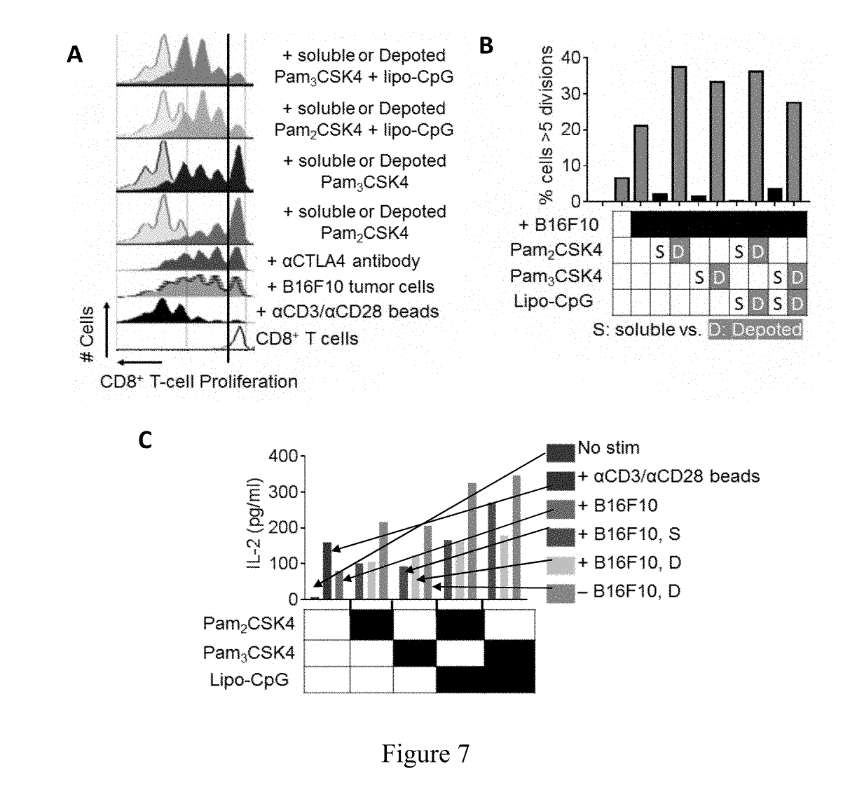

FIG. 7 shows lipo-TLR9 ligand and TLR2 ligand combination-depoted into murine immune cells to overcome suppressive tumor cell signaling. A) Flow cytometry histograms showing CFSE dilution of CD8⁺ T-cells after 3-day stimulation with ratios of 1:1 αCD3/CD28 beads and 1:5 B16F10 tumor cells (blue text). T-Cells were cultured with 10 μg/ml of soluble Pam2CSK4 or Pam3CSK4 either alone or in combination with 5 μM of soluble lipo-CpG (solid fill), or depoted with the same respective combinations (tinted fill). B) Quantification of percent of CD8⁺ T-cells that divided >5 times compared to undivided CD8⁺ T-cells as measured by CF SE dilution from flow cytometry. C) IL-2 production from CD8⁺ T-cell supernatents after 3 days of proliferation. n=1 independent experiment.

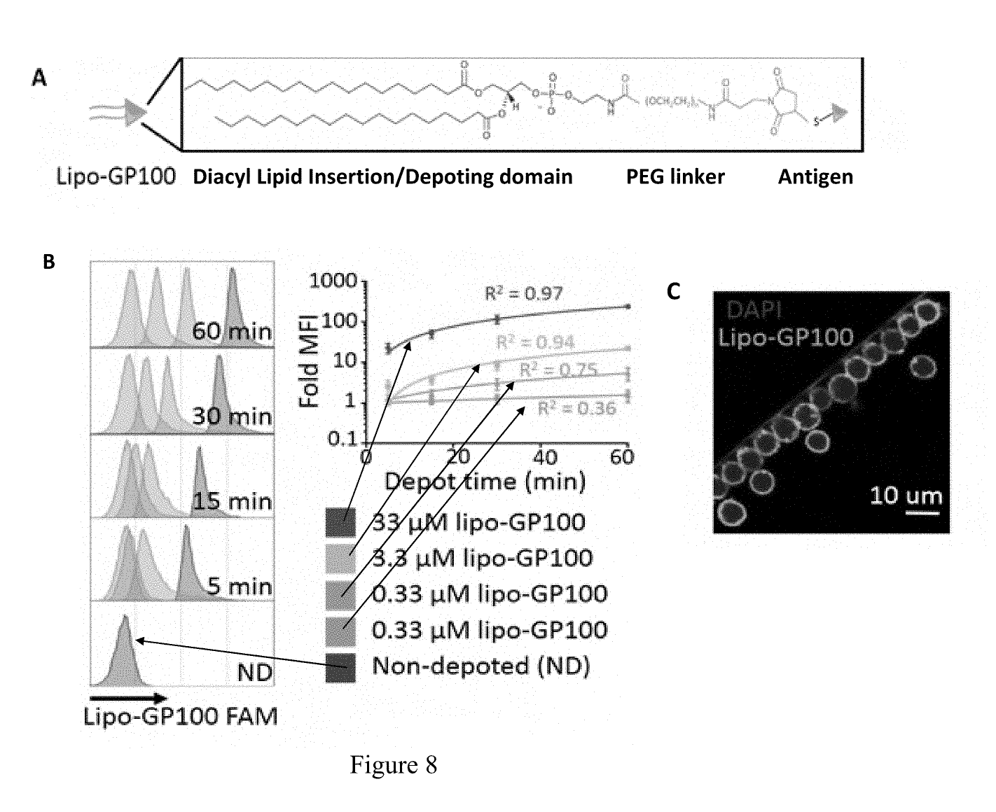

An diacyl (C18) lipid-conjugated ligands (lipo-ligands) was engineered that can be depoted into cell membranes in a time- and concentration-dependent fashion for precise and robust delivery up to 241.5±9.6 (mean±SD) fold increase compared to nondepoted cells. FIG. 8 shows an diacyl (C18) lipid-conjugated ligands (lipo-ligands) that was engineered can be depoted into cell membranes in a time- and concentration-dependent fashion for precise and robust delivery up to 241.5±9.6 (mean±SD) fold increase compared to nondepoted cells. It was observed that efficient and prolonged (>16 hours) antigen presentation of the H-2Db-restricted melanoma peptide GP100₂₅₋₃₃ (KVPRNQDWL) (SEQ ID NO: 2) (lipo-GP100) from B-cells to cognate CD8+ T-cells. FIG. 8 shows A) Structure of lipo-PEG-GP100 (lipo-GP100). B) Representative histograms of fluorescein-labeled (FAM-labeled) lipo-GP100 fluorescence on B-cell surface as functions of depot time and lipo-GP100 concentration measured by flow cytometry. Median fluorescence intensity (MFI) of lipo-GP100 FAM fold increase on B-cells from non-depoted B-cells by flow cytometry. C) MFI of lipo-GP100 FAM fold increased on B-cells from non-depoted B-cells as functions of cell density and reaction volume scaling. n=3 independent samples. D) Microscopy image of B-cells depoted with lipo-GP100 FAM.

This ex vivo delivery strategy was further validated by depoting T-cells with di- and tri-palmitoyl lipid-conjugated TLR2 agonists: Pam2CSK4 and Pam3CSK4, to demonstrate enhanced activation of peripheral APCs as well as increased T-cell proliferation in immunosuppressive environments.

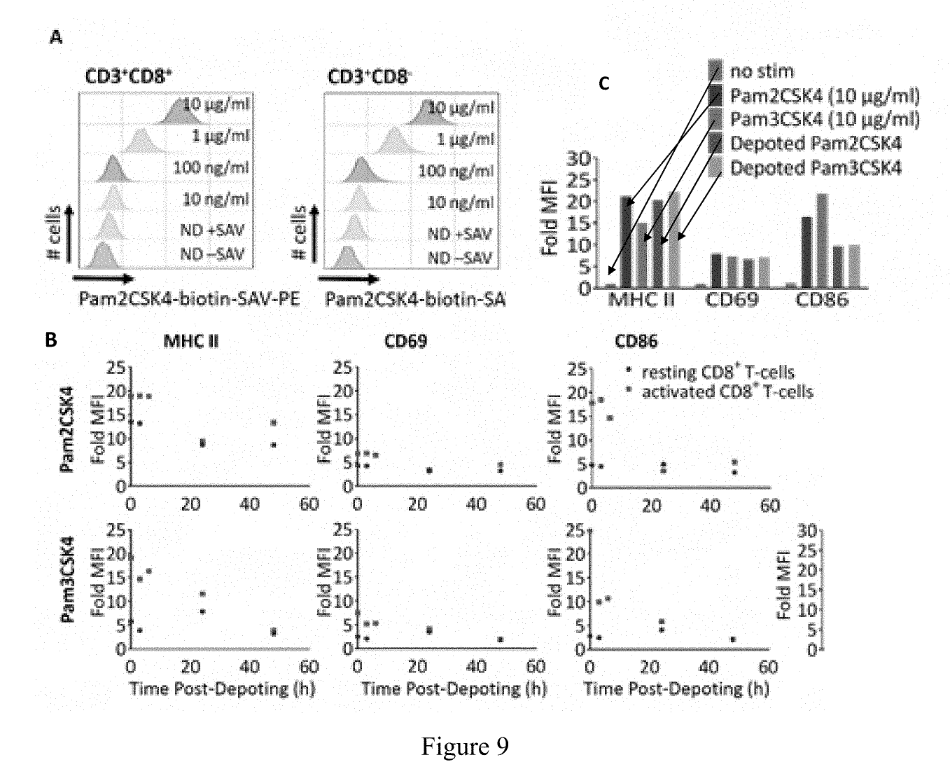

FIG. 9 shows TLR2 ligand depoted into CD3+ T-cells and delivered paracrine signaling. A) Pam2CSK4-biotin at indicated concentrations was depoted into CD3+CD8+ or CD3+CD8− T-cells and stained with streptavidin (SAV) PE as determined by flow cytometry. B) Paracrine signaling of resting vs Concanavalin A/IL-7-activated TLR2 ligand-depoted CD8+ T-cells. Activated CD8+ T-cells were isolated from splenocytes cultured in 2 μg/ml of Concanavalin A and 10 ng/ml of IL-7 for 2 days. TLR2 ligand was depoted and processed by CD8+ T-cells for 0 h, 3 h, 6 h, 24 h, or 48 h, before T-cells were chemically fixed and cultured for 2 days with bystander B-cells to determine fold MFI intensity of MHC II, CD69, and CD86 (C).

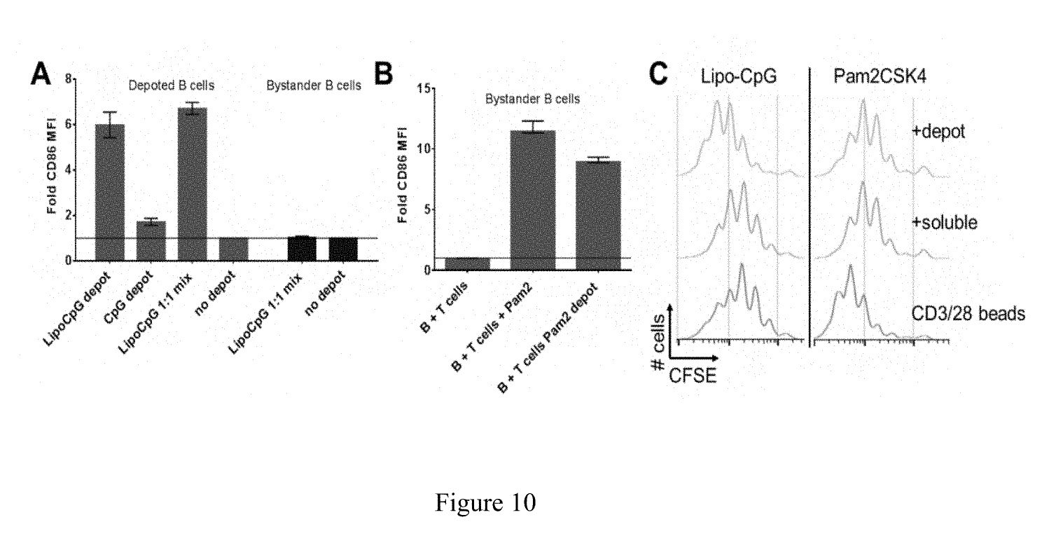

FIG. 10 shows lipo-Avs depoted into B and T cells delivers autocrine and paracrine stimulation. CD45.1+ B cells were depoted with lipo-CpG, CpG, or nothing. These were cultured alone or 1:1 mixed with bystander (no depot) CD45.1− B cells. B cell activation was shown as fold intensity CD86 measured by flow cytometry after 3 d. B) CD8+ T cells were depoted with Pam2CSK4 or nothing, and then co-cultured 1:1 with bystander CD45.1+ B cells for 3 d. B cell activation was measured by CD86 and compared to a continuous exposure Pam2CSK4 control. C) Representative histograms showing CFSE dilution (proliferation) of CD8+ T cells after 3 d stimulation with 1:1 anti-CD3/28 beads. Where indicated, cells were depoted or cultured with continuous soluble lipo-CpG (left) or Pam2CSK4 (right). Bars=median, whiskers=range; n=3.

As stated above, it was found that lipo-CpG can provide autocrine signaling depots to B and T cells with high specificity. B cells depoted with lipo-CpG had sustained activation up to 3 d (FIG. 10 A). When culturing depoted B cells with equal numbers of resting B cells, only depoted cells were activated. This is consistent with the endosomal location of TLR9, requiring that CpG be internalized for signaling, demonstrating exquisite stability of membrane depoting. The ability of a surface sensed lipo-Av to activate neighboring cells was also tested, using Pam2CSK4 which engages surface TLR2. Depoting Pam2 into CD8+ T cells and co-culturing them with resting B cells enabled trans signaling and activation of B cells for 3 days (FIG. 10B), nearly matching continuously exposed control. Lipo-CpG depoting also enhanced CD8+ T cell proliferation in response to αCD3/28 beads, while Pam2CSK4 did not (FIG. 10C). This demonstrated that lipo-CpG could enhance CD8+ T cell proliferation via autocrine signaling, while the surface-sensed Pam2 could not to T cells. These data demonstrate conclusively that lipo-Avs can provide both autocrine and paracrine signaling if using a rational Av. The sustained duration of these effects could significantly enhance B-APC and T effector functions of CBTs in vivo.

Drug delivery to cells has consistently been a major barrier to cell engineering. The preliminary data demonstrated the feasibility and flexibility of using lipid tail insertion into plasma membranes as a simple, rapid, efficient method for delivering biomolecules onto and into cells. This method is inspired by natural glycophosphatidylinositol (GPI)-anchored proteins where GPI inserts itself into plasma membranes. It has been shown herein that both diacyl and triacyl TLR2 ligands, Pam2CSK4 and Pam3CSK4, respectively, can be rapidly inserted into cell membranes.

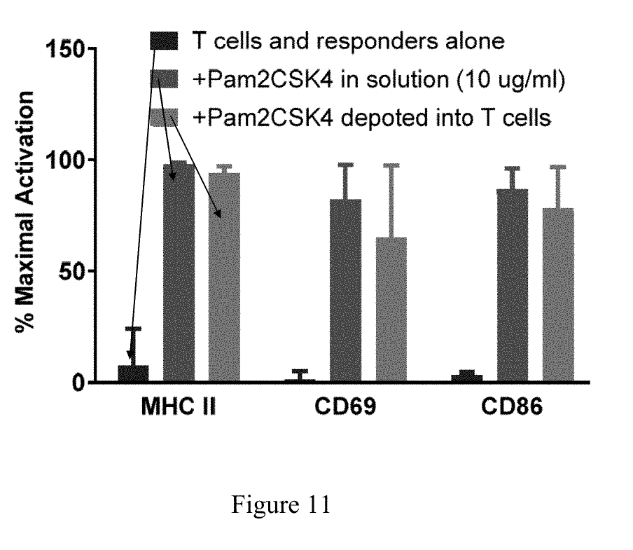

After determining that depoting was a robust process, the function of depoted Pam2CSK4 to activate neighboring immune cells was tested. Depoted T cells were co-cultured with responders (B cells) and the expression of activation markers was determined after 2 days of co-culture at a 1:1 T cell:B cell ratio. It was determined that depoted cells activated neighboring responder cells nearly equivalent to high doses of Pam2CSK4 in solution (FIG. 11) and that this activity persists for multiple days (data not shown). These data indicate that depoted T cells can effectively stimulate bystander immune cells with near equal magnitude to much higher soluble ligand doses. This suggests depoted T cells can deliver highly effective “shock” signals.

In FIG. 11, Pan T cells were isolated from mice and either left alone (blue, red) or depoted with Pam2CSK4 for 1 h (green). T cells were then co-cultured with mouse B cells as a TLR2-responder population. Pam2CSK4 in solution was used as a positive control (red). Expression levels of activation markers were analyzed by flow after 2 days and fluorescence intensity normalized to B cells activated with polyclonal stimulus. Data are shown as median±95% confidence interval (n=3).

The proliferative capacity of depoted CD8+ T cells was evaluated after 3 days of coculture with αCD3/28 beads in the presence or absence of immunosuppressive tumor cells. T cells with tumor cells showed impaired proliferation compared to T cells alone (FIG. 14B, rows 2 vs. 1). The preliminary data showed that T cells depoted with either Pam2CSK4 or Pam3CSK4 recovered proliferative capacity in the presence of tumor cells (FIG. 14B, rows 3-4), and their proliferation was comparable to activated T cells in the absence of tumor cells (FIG. 14B, row 1). This suggests that depoting into cell membranes provides enhanced per-molecule activity, and may provide advantages to CTLs in “shock and kill” scenarios in multiple ways.

The present invention shows enhanced function of immune cells depoted with umethylated CpG DNA (lipo-CpG) by targeting endosomal toll-like receptor 9 (TLR9) while minimizing undesirable TLR9 targeting on bystander cells.

The major barrier to curing HIV infection is the presence of latently infected cells. These virus reservoirs persist even during and after therapy in a quiescent state essentially invisible to the immune system. “Shock and kill” is a promising approach that has emerged for HIV eradication. In this paradigm, agents are delivered to “shock” latently infected cells to reactive latent HIV and express viral proteins, along with targeting by endogenous or engineered effector cells such as cytotoxic T lymphocytes (CTLs) to deliver the “kill” and eliminate these reservoirs. Most “shock and kill” studies have focused on eliminating the resting memory CD4+ T cell reservoir. Myeloid-derived tissue-resident cells such as macrophages and microglia are long-lived populations that have also been shown to harbor latent virus. These cells are seeded throughout tissues such as the spleen, gut, and brain rapidly after HIV infection. They also serve as viral reservoirs that contribute to viral replication and rebound after cessation of antiretroviral (ARV) therapy. Importantly, systemic administration of any agent is inefficient, with only a fraction of injected dose penetrating tissues and the majority of intravenously administered doses filtered and metabolized by the liver and kidney. Thus use of the present delivery device will overcome the shortcomings of systemic administration.

Given the above, a “shock and kill” regimen would benefit from these properties: 1) reactivation of both T cell and myeloid reservoirs, 2) distribution of “shock” agents throughout tissue sites, and 3) enhanced “kill” activity of CTLs against HIV-infected cells. Optimizing these regimens needs to include coordination of the action of “shock” drugs with the “kill” activity from CTLs and activation-induced enhancement of cytotoxic CTL activity, increasing overall therapeutic efficacy while reducing toxicity.

The TLR2 ligand Pam3CSK4 has been identified as an effective “shock” agent in resting memory CD4+ T cells that can also enhance CTL “kill” activity, but the potential for reactivation and killing of primary HIV-infected myeloid cells has not been fully explored. Notably, as shown above, the TLR2 ligand Pam3CSK4 and has been inserted into a cell with the delivery device of the present invention. In the present invention, a macrophage-inclusive approach for HIV eradication is evaluated for leveraging a rapid platform for engineering CTLs to optimize “shock and kill” by simultaneously shocking T cell and myeloid reservoirs, increasing tissue penetrance and colocalization of drug activity, and developing novel CTL-enhancing drugs.

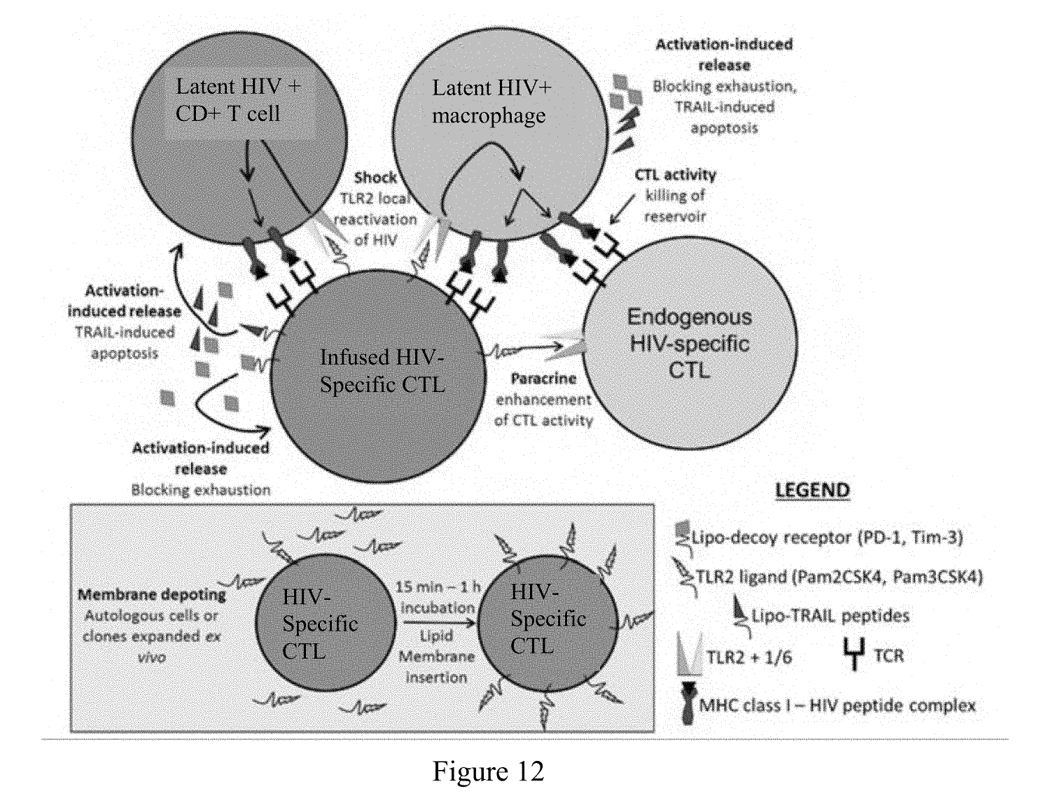

Lipid-tailed biomolecules are introduced into CTLs (hereafter termed “depoting”) for enhanced “shock and kill” regimens as shown in FIG. 12. Advantages of this approach include rapid and efficient cell engineering, transient non-genetic modification reducing potential toxic and mutagenic side effects, colocalization and coordination of “shock” and “kill” drug effects, future potential for enhanced tissue distribution of drugs, and improved CTL efficacy against both macrophage and CD4+ T cell viral reservoirs.

That which is claimed is:

1. An ex vivo method of introducing a biomolecule cargo into a cell without mechanical disturbance of a cell membrane of the cell, the method comprising:
contacting the cell with lipid-tailed biomolecule, wherein the lipid-tailed biomolecule comprises:
(a) a lipid component;
(b) a biomolecule cargo for delivery into a cell; and
(c) a linker positioned between and connecting the lipid component and the biomolecule cargo to form the lipid-tailed molecule; and
maintaining the cell in a culture medium a sufficient time for the lipid component of the lipid-tailed molecule to blend with lipids positioned on the cell membrane without mechanical disturbance of a cell membrane and insertion of the biomolecule cargo into the cell. This ex vivo method provides for expansion of the cells with the inclusion of the biomolecule cargo.

2. The ex vivo method of claim 1 wherein the biomolecule cargo is an immunomodulatory compound, immunoregulatory compound, a molecular adjuvant, nucleosides, nucleotides or oligonucleotides, proteins or peptides.

3. The ex vivo method of claim 1, wherein the lipid component is selected from the group consisting of fatty acids with aliphatic tails of 8-30 carbons, linear saturated and unsaturated fatty acids, branched saturated and unsaturated fatty acids, fatty acids derivatives, fatty acid esters, fatty acid amides, fatty acid thioesters, diacyl lipids, Cholesterol, Cholesterol derivatives, steroid acids and combinations thereof.

4. The ex vivo method of claim 1, wherein the lipid component is selected for targeting specific intracellular locations.

5. The ex vivo method of claim 1 wherein the cell is selected from the group consisting of a lymphokine-activated killer cell, a dendritic cell, a monocyte, a B cell, a T-cell, a natural killer cell, a neutrophil, an eosinophil, a basophil, a mast cell, a keratinocyte, an endothelial cell, an islet cell, a fibroblast, an osteoblast, a chondrocyte, a muscle cell, a stem cell and a neural cell.

6. The ex vivo method of claim 1, wherein the linker is selected from two or more ethylene glycol (EG) units, cell-triggered linkers, tissue or organ response linkers, degradable linkers, or reactable linkers.

7. The ex vivo method of claim 6, wherein the two or more ethylene glycol units in in an amount from about 20 to 80 units.

8. The ex vivo method of claim 1, wherein the lipid tail is coupled to the linker by a bond selected from the group consisting of an ester bond linkage, amide bond linkage, thioester bond linkage, or combinations thereof.

9. The ex vivo method of claim 8, wherein the bond between the lipid tail and the linker/cargo is a covalent bond that is a cleavable or non-cleavable bond.

10. The ex vivo method of claim 9, wherein the cleavable bond is a disulfide bond, acid-cleavable linkage, ester bond, anhydride bond, biodegradable bond, or enzyme-cleavable linkage and the non-cleavable bond is an amide bond or phosphate bond.

11. A lipid-tailed biomolecule comprising
(a) a lipid component;
(b) a biomolecule cargo for delivery into a cell; and
(c) a linker positioned between and connecting the lipid component and the biomolecule cargo to form the lipid-tailed biomolecule.

12. The lipid-tailed biomolecule of claim 11 wherein the biomolecule cargo is an immunomodulatory compound, immunoregulatory compound, a molecular adjuvant, nucleosides, nucleotides or oligonucleotides, proteins or peptides.

13. The lipid-tailed biomolecule of claim 11, wherein the lipid component is selected from the group consisting of fatty acids with aliphatic tails of 8-30 carbons, linear saturated and unsaturated fatty acids, branched saturated and unsaturated fatty acids, fatty acids derivatives, fatty acid esters, fatty acid amides, fatty acid thioesters, diacyl lipids, Cholesterol, Cholesterol derivatives, steroid acids and combinations thereof.

14. The lipid-tailed biomolecule of claim 11, wherein the lipid component is selected for targeting specific intracellular locations.

15. The lipid-tailed biomolecule of claim 11, wherein the cell is selected from the group consisting of a lymphokine-activated killer cell, a dendritic cell, a monocyte, a B cell, a T-cell, a natural killer cell, a neutrophil, an eosinophil, a basophil, a mast cell, a keratinocyte, an endothelial cell, an islet cell, a fibroblast, an osteoblast, a chondrocyte, a muscle cell, a stem cell and a neural cell.

16. The lipid-tailed biomolecule of claim 11, wherein the linker is selected from two or more ethylene glycol (EG) units, cell-triggered linkers, tissue or organ response linkers, degradable linkers, or reactable linkers.

17. The lipid-tailed biomolecule of claim 16, wherein the two or more ethylene glycol units in in an amount from about 20 to 80 units.

18. The lipid-tailed biomolecule of claim 11, wherein the lipid tail is coupled to the linker by a bond selected from the group consisting of an ester bond linkage, amide bond linkage, thioester bond linkage, or combinations thereof.

19. The lipid-tailed biomolecule of claim 18, wherein the bond between the lipid tail and the linker/cargo is a covalent bond that is a cleavable or non-cleavable bond.

20. The lipid-tailed biomolecule of claim 19, wherein the cleavable bond is a disulfide bond, acid-cleavable linkage, ester bond, anhydride bond, biodegradable bond, or enzyme-cleavable linkage and the non-cleavable bond is an amide bond or phosphate bond.

21. A method of treating a patient for an illness, the method comprising:
preparing a lipid-tailed biomolecule for entry into isolated and/or removed cells from the patient:
contacting the isolated and/or removed cells with the lipid-tailed biomolecule, wherein the lipid-tailed biomolecule comprises:
(a) a lipid component;
(b) a biomolecule cargo for delivery into a cell; and
(c) a linker positioned between and connecting the lipid component and the biomolecule cargo;
maintaining the isolated and/or removed cells in an ex vivo culture medium a sufficient time for the lipid component of the lipid-tailed molecule to blend with lipids positioned on the cell membrane without mechanical disturbance of a cell membrane and insertion of the biomolecule cargo into the isolated and removed cells to form activated cells; and
administering and reintroducing an effective amount of the activated cells into the patient.

22. The method of claim 21, wherein the illness is cancer, viral infection, autoimmune disease or alloimmune disease.

23. The method of claim 21 wherein the biomolecule cargo is an immunomodulatory compound, immunoregulatory compound, a molecular adjuvant, nucleosides, nucleotides or oligonucleotides, proteins or peptides.

24. The method of claim 21, wherein the lipid component is a diacyl lipid or triacyl lipid having carbon chains between about 12 and 22.

25. The method of claim 21 wherein the cell is selected from the group consisting of a lymphokine-activated killer cell, a dendritic cell, a monocyte, a B cell, a T-cell, a natural killer cell, a neutrophil, an eosinophil, a basophil, a mast cell, a keratinocyte, an endothelial cell, an islet cell, a fibroblast, an osteoblast, a chondrocyte, a muscle cell, a stem cell and a neural cell.

26. The method of claim 21, where the isolated and/or removed cells in the ex vivo culture medium are maintained at a temperature from between 20 to 45° C. and a pH of from about 6 to 7.5.

27. The method of claim 26, wherein the ex vivo culture medium further nutrients selected from carbon, hydrogen, oxygen, nitrogen, sulphur and phosphorus.

28. The method of claim 21, wherein the lipid component is selected from the group consisting of fatty acids with aliphatic tails of 8-30 carbons, linear saturated and unsaturated fatty acids, branched saturated and unsaturated fatty acids, fatty acids derivatives, fatty acid esters, fatty acid amides, fatty acid thioesters, diacyl lipids, Cholesterol, Cholesterol derivatives, steroid acids and combinations thereof.

29. The method of claim 21, wherein the lipid component is selected for targeting specific intracellular locations.

30. The method of claim 21, wherein the linker is selected from two or more ethylene glycol (EG) units, cell-triggered linkers, tissue or organ response linkers, degradable linkers, or reactable linkers.

31. The method of claim 30, wherein the two or more ethylene glycol units in in an amount from about 20 to 80 units.

32. The method of claim 21, wherein the lipid tail is coupled to the linker by a bond selected from the group consisting of an ester bond linkage, amide bond linkage, thioester bond linkage, or combinations thereof.

33. The method of claim 32, wherein the bond between the lipid tail and the linker/cargo is a covalent bond that is a cleavable or non-cleavable bond.

34. The method of claim 33, wherein the cleavable bond is a disulfide bond, acid-cleavable linkage, ester bond, anhydride bond, biodegradable bond, or enzyme-cleavable linkage and the non-cleavable bond is an amide bond or phosphate bond.

35. Therapeutic cells for the study and treatment of cancer, viral, autoimmune and alloimmune diseases and disorders produced by the method of claim 1.


SEQUENCE LISTING (2 sequences)

SEQ ID NO: 1 - DNA, 20 bp, Artificial Sequence
  Synthetic Construct
tccatgacgttcctgacgtt

SEQ ID NO: 2 - protein, 9 aa, Artificial Sequence
  Synthetic Construct
KVPRNQDWL